(19) United States
(12) Patent Application Publication
(10) Pub. No.: US 2009/0145446 A1
(43) Pub. Date: Jun. 11, 2009

(54) Method for detecting at least partial medical device disengagement from a patient

(76) Inventor: Paul G. DeDecker, Clinton Twp, MI (US)

Correspondence Address: DELPHI TECHNOLOGIES, INC., M/C 480-410-202, PO BOX 5052, TROY, MI, 48007, US

(21) Appl. No.: 11/999,962

(22) Filed: Dec. 7, 2007

(51) Int. Cl. A61M 39/00

(52) U.S. Cl. 128/899

(57) ABSTRACT

A method for detecting at least partial medical device disengagement from a patient includes inserting the device in the patient so a Hall effect sensor connected to the device is positioned at an initial location substantially proximate to one of a plurality of magnetically charged components. Each of the components is removably attached to the patient, and is positioned a predetermined distance from another component. A magnetic field indicative of the one of the plurality of magnetically charged components is detected with the sensor. The sensor monitors for a failure to detect the magnetic field. In response to the failure detection, the sensor monitors for a subsequent detection of another magnetic field indicative of another of the plurality of magnetically charged components. The subsequent detection is associated with movement of the medical device substantially equal to the predetermined distance between the one and the other of the components.

### BACKGROUND

The present disclosure relates generally to sensors, and more particularly to a method and system for detecting, with a sensor, at least partial medical device disengagement from a patient.

Medical devices (non-limiting examples of which include needles and catheters) used to deliver fluids to patients may move relative to the point of insertion. Such movement may be due, at least in part, to patient movement or to small forces acting on the tubing connected to the medical device. Device movement may potentially be problematic for various reasons.

Systems have been developed to affix the medical devices to the patient, including utilizing tape or other adhesives to secure the medical device to the patient. Such a securing method, however, may not prevent disengagement of the medical device from the patient, and generally does not provide notice in the event of a partial or complete disengagement.

Systems have also been developed to monitor medical device disconnect. Such systems include optical sensors and/or electrical signal sensors. Optical sensors may be subject to sensitivity variations due, at least in part, to ambient lighting, light path blockages, and surface contamination. Electrical signal sensors measure changes in, for example, impedance, resistance or capacitance to detect medical device disengagement. The sensitivity of such a system is balanced to minimize the occurrence of false alarms while providing adequate sensitivity to detect actual disconnect occurrences.

### SUMMARY

A method for detecting at least partial medical device disengagement from a patient is disclosed. The method includes inserting the medical device in the patient so that a Hall effect sensor operatively connected to the medical device is operatively positioned at an initial location that is substantially proximate to one of a plurality of magnetically charged components that are removably attached to the patient. Each of the plurality of magnetically charged components is positioned a predetermined distance from another of the plurality of magnetically charged components. The sensor is used to detect a magnetic field that is indicative of the one of the plurality of magnetically charged components. The sensor is also used to monitor for 1) a failure to detect such a magnetic field, and 2) a subsequent detection of another magnetic field indicative of another of the plurality of magnetically charged components in response to the failure to detect the magnetic field indicative of the one of the plurality of the magnetically charged components. The subsequent detection is associated with movement of the medical device substantially equal to the predetermined distance between the one of the plurality of magnetically charged components and the other of the plurality of the magnetically charged components.

### BRIEF DESCRIPTION OF THE DRAWINGS

Features and advantages of embodiments of the present disclosure will become apparent by reference to the following detailed description and drawings, in which like reference numerals correspond to similar, though perhaps not identical components. Reference numerals having a previously described function may or may not be described in connection with other drawings in which they appear.

FIG. 1 is a schematic cutaway view of an embodiment of a device configured to detect at least partial disengagement of a medical device from a patient; and

FIG. 2 is a flow diagram depicting an embodiment of a method for detecting at least partial medical device disengagement from a patient.

### DETAILED DESCRIPTION

Embodiment(s) of the method and device disclosed herein advantageously include a Hall effect sensor configured to detect, when proximate thereto, a magnetic field of one or more of a plurality of magnetically charged components spaced at predetermined intervals from a medical device point of insertion located on a patient. The Hall effect sensor is operatively connected to the medical device (such as a needle or catheter), so that the sensor moves with the medical device. If the device disengages and moves proximate to one or more of the magnetically charged components, the Hall effect sensor advantageously detects the magnetic fields given off by those magnetically charged component(s). The distance traveled (relative to the point of insertion) by the needle and sensor may advantageously be determined via the detected magnetic fields.

Initially, the sensor is substantially aligned with one of the magnetically charged components (e.g., the component nearest the device point of insertion), whereby the sensor detects a magnetic field indicative of that component. If the medical device disengages from the patient so that the sensor is no longer proximate to that component, the sensor will lose detection of the component. If the medical device disengages even further, the sensor will detect the next magnetically charged component in alignment. As such, the Hall effect sensor will cyclically detect and fail to detect magnetic fields of respective magnetically charged components as the medical device becomes more disengaged from the patient. Such alternating detections and failures to detect indicate that the medical device has at least partially disengaged.

In one embodiment, the device advantageously includes at least one disposable component (e.g., the magnetically charged components), and at least one reusable component (e.g., the Hall effect sensor). In another embodiment, the sensor is disposable, and the magnetically charged components are reusable. It is to be understood that the entire device may be disposable, or it may be reusable. It is to be further understood that any combination of disposable and reusable components may be used to form the device.

Generally, embodiments of the method and system disclosed herein provide a relatively simple and low-cost means to monitor the location of a medical device that has been inserted in a patient. The method and system disclosed herein may also advantageously provide means to notify a user (i.e., patient or caretaker) of at least partial disengagement of the medical device from the patient.

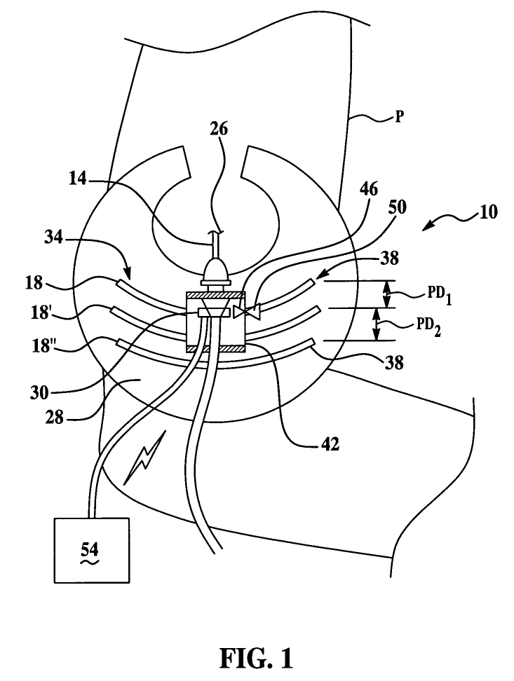

Referring now to FIG. 1, an embodiment of the device 10 configured to detect at least partial disengagement of a medical device 14 from a patient P is depicted. In this embodiment, the device 10 is a stand alone unit. The device 10 includes a plurality of magnetically charged components 18, 18′, 18″ that is operatively and removably attachable to the patient P. Each of the magnetically charged components 18, 18′, 18″ is positioned a predetermined distance PD₁, PD₂ from another of the magnetically charged components 18, 18′, 18″. As shown in FIG. 1, the first magnetically charged component 18 is positioned a predetermined distance PD₁ from the second magnetically charged component 18′, and the second magnetically charged component 18′ is positioned a distance PD₂ from the third magnetically charged component 18″. As such, the distance between the first and third magnetically charged components 18, 18″ is approximately the sum of the two predetermined distances PD₁, PD₂. The predetermined distances PD₁, PD₂ between the respective components 18, 18′, 18″ may be the same or different. In one non-limiting example, each predetermined distance PD₁, PD₂ is equal to or less than about 1.3 cm (0.5 inches).

In an embodiment, the device 10 includes three magnetically charged components 18, 18′, 18″. It is to be understood, however, that the device 10 may include as little as two magnetically charged components 18, 18′, 18″, and as many as are desirable. Generally, the number of magnetically charged components 18, 18′, 18″ may correspond to the length of the medical device 14. In a non-limiting example, eight magnetically charged components 18,18′, 18″ at about 0.5 inch spacing may be desirable for a relatively long medical device 14. If shorter spacing were used with the same size medical device 14, then more than eight components 18, 18′, 18″ may be used.

In an embodiment, each magnetically charged component 18, 18′, 18″ has two longitudinal ends 34, 38. The components 18, 18′, 18″ may have any shape, geometry, or configuration that is suitable at least for adhering to the patient P, and for having the sensor 30 and medical device 14 move in proximity thereto.

During use, the medical device 14 is inserted into the patient P at a point of insertion 26. The medical device 14 may deliver a fluid, such as blood or medication, to the patient P. As non-limiting examples, the medical device 14 may be a needle or a catheter. In an embodiment, the medical device 14 is utilized during dialysis.

The magnetically charged components 18, 18′, 18″ are removably fixed to the patient P at a position relative to the point of insertion 26. It is to be understood that the components 18, 18′, 18″ may be attached to the patient P via any suitable means. In an embodiment (as shown in FIG. 1), the magnetically charged components 18, 18′, 18″ are affixed to an adhesive bandage 28, which is capable of adhering to the patient's skin. In another embodiment, the magnetically charged components 18, 18′, 18″ are affixed to a band (e.g., an armband, legband, etc.). The band may be formed, for example, from elastic, rubber, or any other suitable material. The band may be secured via a hook, a snap, a button, a hook-and-loop fastener, ties, an adhesive, or combinations thereof. It is to be understood that the magnetically charged components 18, 18′, 18″ may be affixed to the bandage 28, the band, and/or the like, by any suitable means. As non-limiting examples, the components 18, 18′, 18″ may be adhered, stitched, laced, embedded, and/or printed on the bandage/band/etc.

As shown in FIG. 1, the Hall effect sensor 30 is affixed to the medical device 14. As a non-limiting example, the sensor 30 may be affixed to the medical device 14 via a clip, a needle clamp, or any other suitable means. The sensor 30 may be battery operated, and may advantageously consume relatively low amounts of power. The sensor 30 may be permanently affixed or removably affixed to the medical device 14. In one embodiment, the sensor 30 is removably affixed to the medical device 14, so the medical device 14 may be disposed of after use, and the sensor 30 may be reused.

The sensor 30 detects the magnetic field of each magnetically charged component 18, 18′, 18″ when located in proximity thereto, or substantially aligned therewith. When the sensor 30 is not proximate to one of the components 18, 18′, 18″, the sensor 30 fails to detect a magnetic field.

The Hall effect sensor 30 may function as a switch, and a circuit that detects closure of the switch may be integrated with or otherwise connected to the sensor 30. When the Hall effect sensor 30 is substantially aligned with one of the magnetically charged components 18, 18′, 18″, the sensor 30 is activated or triggered, which is detectable by the circuit. As such, if the initial position of the sensor 30 is substantially proximate to one of the magnetically charged components 18, 18′, 18″ (e.g., component 18 in FIG. 1), the switch will initially be active (i.e., the switch is closed). If moved from the initial position and out of proximity of the component 18, a failure to detect the field of the component 18 will result, and the switch will become inactive (i.e., open). Subsequent detections and failures to detect the other components 18′, 18″ are noted by the circuit as subsequent activations and inactivations of the switch, respectively.

The number of switch activations/triggers is proportional to the distance traveled by the medical device 14, and, as such, may be utilized to calculate the disengagement distance of the device 14. Additionally, a loss of initial activity, followed by one or more triggers (i.e., activations) may provide a high degree of assurance that the medical device 14 has become at least partially disengaged.

It is to be understood that the device 10 has a detection range where the sensor 30 is capable of detecting the field of the magnetically charged components 18, 18′, 18″. More specifically, the sensor 30 is capable of detecting the field of the magnetically charged component(s) 18, 18′, 18″ when it is precisely aligned therewith, and, in some embodiments, when it is located within a predetermined alignment distance from the component(s) 18, 18′, 18″. The predetermined alignment distance may be, for example, when the sensor 30 is located within 0.25 mm, 0.5 mm, or 1 mm, etc., of the component 18, 18′, 18″. It is to be understood that the predetermined alignment distance extends in the direction of engagement/disengagement. As non-limiting examples, the actual distance that the sensor 30 may travel while still detecting a particular magnetically charged component 18, 18′, 18″ may be 0.5 mm, 1 mm, or 2 mm, etc. In a non-limiting example, the alignment distance is 0, whereby the sensor 30 detects the component 18, 18′, 18″ when precisely aligned therewith.

The magnetically charged components 18, 18′, 18″ may be positioned so that adjacent components 18, 18′, 18″ have opposite polarities (e.g., north, south, north, or south, north, south). In an embodiment, the component 18 has one of the north or south pole directly adjacent the patient's skin, while the other of the south or north pole is positioned away from the patient's skin. In such an embodiment, the magnetically charged component 18′ nearest the component 18 has the other of the south or north pole directly adjacent the patient's skin and the north or south pole positioned away from the patient's skin. In still another embodiment, component 18 has the north or south pole located at one longitudinal end 34 of the component 18 while the other of the south or north pole is located at the opposite longitudinal end 38. In such an embodiment, the nearest component(s) 18′ have the other of the south or north pole located at the one longitudinal end 34 while the other of the north or south pole is located at the opposite longitudinal end 38.

It may also be desirable to form a vertically tall field (the height of which is measured in relation to the patient's skin) that is narrow in the plane of the bandage 28. Without being bound to any theory, it is believed that this embodiment substantially maximizes the mis-alignment tolerance of the sensor 30. The magnetic components 18, 18′, 18″ in this embodiment are self-contained with both poles (north and south) stacked on top of each other in the plane of the bandage 28.

The sensor 30 responds to any sufficiently strong magnetic field to which it is exposed. As such, the sensor 30 may be activated regardless of the polarity to which it is exposed.

The sensor 30 is capable of transmitting the detected magnetic field as a digital signal. It is to be understood that the sensor 30 may transmit no signal indicating a lack of the detected magnetic field when the sensor 30 is not in proximity to one of the magnetically charged components 18, 18′, 18″.

Referring to the specific example of FIG. 1, when the medical device 14 is initially inserted in the patient P, the Hall effect sensor 30 is substantially aligned with the first magnetically charged component 18 such that the sensor 30 detects the magnetic field of the first component 18. If the medical device 14 becomes at least partially disengaged from the patient P a distance that is less than the predetermined distance PD₁ and greater than the alignment distance, the sensor 30 fails to detect the magnetic field of the first component 18. The sensor 30 (or a processor 32 in operative communication therewith) associates the failure to detect the magnetic field with movement of the medical device 14, where the movement is less than the predetermined distance PD₁.

As the medical device 14 becomes further disengaged, the sensor 30 may come into the proximity of another magnetically charged component 18′, 18″. As shown in FIG. 1, if the medical device 14 is disengaged a distance approximately equal to the predetermined distance PD₁, the sensor 30 detects the second magnetically charged component 18′. The sensor 30 associates such detection with disengagement of the medical device 14, where the distance of disengagement is substantially equal to the predetermined distance PD₁.

Still referring to the embodiment depicted in FIG. 1, if the Hall effect sensor 30 fails to detect the magnetic field of the second magnetically charged component 18′ (i.e., fails to detect the second component 18′ after detection thereof), the sensor 30 associates the failure with further disengagement of the medical device 14. Such a failure indicates that the medical device 14 and sensor 30 have moved out of alignment with the second magnetically charged component 18′. Generally, this failure to detect indicates that the distance moved is greater than the predetermined distance PD, between the components 18, 18′, but less than the predetermined distance PD₂ between the components 18′, 18″.

If the sensor 30 detects the third magnetically charged component 18″, the sensor 30 associates such a detection with disengagement of the medical device 14 substantially equal to the sum of the predetermined distances PD₁, PD₂. Yet further, if the sensor 30 loses the detection of the third component 18″, the sensor 30 associates the failure to detect the third component 18″ with movement of the medical device 14 beyond the sum of predetermined distances PD₁, PD₂.

As such, it is to be understood that the distance of disengagement (i.e., total distance moved by the medical device 14 as measured from the initial position) may be calculated by adding the predetermined distances PD₁, PD₂ together after the component 18, 18′, 18″ associated with that distance PD₁, PD₂ has been detected and then undetected.

It is to be further understood that the processor 32 may be embodied in the Hall effect sensor 30 or may be a separate component in operative communication therewith. Generally, the sensor 30 detects the magnetic field or lack thereof, and transmits signals to the processor 32. The signals and timing between received signals are analyzed by the processor 32. It is to be understood that the number of signals and the timing between the signals may be equated to a level of alarm. Generally, the alarm level is escalated as the number of signals increases and/or as the time between receiving signals decreases.

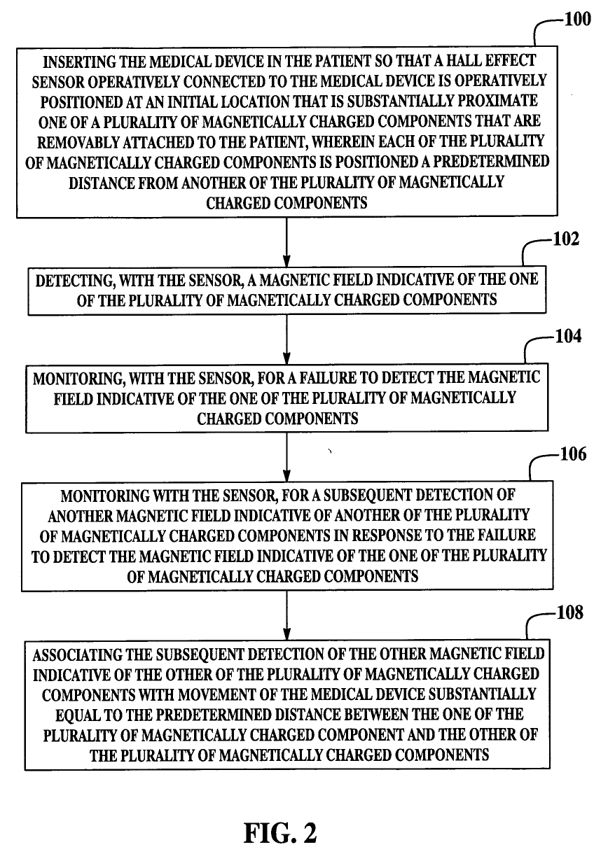

Referring now to FIG. 2, an embodiment of a method for detecting at least partial medical device 14 disengagement from a patient P is depicted. The method includes inserting the medical device 14 in the patient P so that the Hall effect sensor 30 (which is operatively connected to the medical device 14) is operatively positioned at an initial location that is substantially proximate to one of the plurality of magnetically charged components 18, as depicted at reference numeral 100. As previously described, each of the plurality of magnetically charged components 18, 18′, 18″ is spaced a predetermined distance PD₁, PD₂ from another of the components 18, 18′, 18″.

The embodiment further includes detecting, with the sensor 30, a magnetic field indicative of the magnetically charged component 18 substantially proximate to the sensor 30, as depicted at reference numeral 102, and monitoring, with the sensor 30, for a failure to detect the magnetic field indicative of the magnetically charged component 18, as depicted at reference numeral 103. Further, the embodiment includes monitoring, with the sensor 30, for a subsequent detection of another magnetic field indicative of another of the plurality of magnetically charged components 18′, 18″ in response to the failure to detect the magnetic field of the component 18, as depicted at reference numeral 104. Yet further, the embodiment includes associating the subsequent detection of the other magnetic field indicative of the other of the magnetically charged component(s) 18′, 18″ with movement of the medical device 14 substantially equal to the predetermined distance PD₁, PD₂ between the one component 18 and the other component 18′, 18″, as depicted at reference numeral 105.

The device 10 may be initialized, whereby the Hall effect sensor 30 is aligned with one of the magnetically charged components 18, 18′, 18″, either automatically or manually. In an embodiment where the initialization is automatic, the device 10, when powered on, begins monitoring for detection of a component 18, 18′, 18″. A user substantially aligns the Hall effect sensor 30 with the first magnetically charged component 18, and maintains the alignment for a predetermined length of time (e.g., less than one second (e.g., substantially instantaneous), ten seconds, thirty seconds, one minute, etc.), which may be user-defined or a default value. If the sensor 30 detects the component 18 for the predetermined length of time, the device 10 assumes initialization and begins monitoring for a failure to detect the component 18. In an embodiment where the initialization is manual, a user substantially aligns the Hall effect sensor 30 with the first component 18, and notifies the device 10 that initialization is complete. As non-limiting examples, the user may notify the device 10 that initialization is complete by providing an input via one or more buttons, switches, knobs, and/or the like, and/or combinations thereof.

To aid in substantially aligning the sensor 30 with the first component 18, an indicator may be placed on the sensor 30 and on the component 18. As illustrated in FIG. 1, the sensor housing 42 includes an arrow 46 (although any suitable indicia may be utilized) that substantially aligns with the arrow 50 on the component 18 when the sensor 30 is substantially aligned with the component 18. In another embodiment, the device 10 is equipped with a light emitting diode (LED), or other indicator, that provides notice to a user when the sensor 30 is substantially aligned with one of the magnetically charged components 18. In another embodiment, the indicator may provide notice to the user when the sensor 30 is substantially aligned with a magnetically charged component 18 having a particular polar alignment.

The device 10 may also provide notice to a user (e.g., patient or caretaker) if the medical device 14 moves beyond a predetermined threshold. The predetermined threshold may be a distance short of complete device 14 disengagement, so that a caretaker and user may be warned prior to the medical device 14 becoming completely disengaged from the patient P. Non-limiting examples of the predetermined threshold are 0.5 cm, 1 cm, 1.5 cm, or 2 cm from the point of insertion 26.

As such, the Hall effect sensor 30 and/or the processor 32 may be in operative communication with an alert system 54. The alert system 54 receives an alert signal from the sensor 30 (or the processor 32) when the determined (or calculated) disengagement of the medical device 14 has exceeded the predetermined threshold. In response to the alert signal, the alert system 54 may emit an alarm. The alarm may be embodied as an audible alarm, a visual alarm, a tactile alarm, and/or the like, and/or combinations thereof.

FIG. 1 depicts a wired alert system 54. In another embodiment not shown in the Figures, the alert system 54 is operatively connected to the sensor 30 and/or processor 32 via a wireless connection. It is to be understood that either configuration, or a combination of both configurations, may be suitable for the device 10.

In the wired embodiment of the alert system 54, a cable electrically connects the sensor 30 to an electronic device (non-limitative examples of which include pagers, computers, personal digital assistants (PDAs), cellular phones, and combinations thereof) and/or a machine (a non-limiting example of which includes a hemodialysis machine) that is operatively connected to the alert system 54. It is to be understood that the cable may electrically connect to a power outlet strip, where the machine and/or the electronic device may be plugged in.

In the wireless embodiment of the alert system 54, radio frequency or infrared means electrically and operatively connect the sensor 30 to the electronic device and/or the machine. In a non-limitative example, a transmitter (not shown) is operatively connected to the sensor 30, and a receiver (not shown) is operatively connected to the electronic device and/or the machine. In another non-limitative example embodiment, the receiver may be positioned in, and electrically connected to, the power outlet strip.

Upon recognition of partial or full disengagement of the medical device 14 from the engaged position, the sensor 30 sends a signal to the alert system 54. In response, the alert system 54 is capable of generating an alarm. As previously stated, non-limitative examples of the alarm include visual alarms, audio alarms, tactile alarms, and/or the like, and/or combinations thereof.

In alternate embodiments, the alarm is capable of sending a signal to the electronic device and/or automatically shutting down the machine.

In an embodiment where the alert system 54 is operatively connected (e.g. via a cable or a wireless connection) with the power strip, the alarm interrupts a main power supply to any machine or device (e.g., the hemodialysis machine) that is plugged into the modified power strip. As such, the machine or device loses power and shuts down. It is to be understood that in this embodiment, the power strip may be modified to include electronics capable of interrupting the power supply, thereby shutting down the machine, upon recognition of the alarm.

In another embodiment not depicted in the figures, the alert system 54 is operatively connected to the electronic device, which is also operatively connected to the machine. In this embodiment, upon recognizing the alarm, the electronic device signals the machine to shut down. Still further, the electronic device may also or alternately be programmed so that the patient P or other person may manually shut down the machine via the electronic device when the alarm is generated. It is to be understood that in this embodiment, the machine may be modified to include electronics capable of shutting down the machine upon recognition of the signal from the electronic device.

It is to be understood that the previous methods of automatic shutdown may be combined such that a safeguard mode is implemented if one of the shutdown mechanisms fails.

In another embodiment of the device 10, the sensor 30 may be attached to the patient P (e.g., via bandage 28), and the magnetically charged components 18, 18′, 18″ may be securely attached to the medical device 14. Generally, any configuration of the medical device 14, sensor 30 and magnetically charged components 18, 18′, 18″ is contemplated herein, as long as relative motion between the medical device 14 and the point of insertion 26 may be monitored using such components.

It is to be understood that the device 10 may be used in conjunction with other sensing methods, such as optical sensing methods, electrical sensing methods, or combinations thereof.

Further, it is to be understood that the terms “engaged/engage/engaging” , in “communication″ with and/or the like are broadly defined herein to encompass a variety of divergent connected arrangements and assembly techniques. These arrangements and techniques include, but are not limited to (1) the direct communication between one component and another component with no intervening components therebetween; and (2) the communication of one component and another component with one or more components therebetween, provided that the one component being “engaged with” the other component is somehow in operative communication with the other component (notwithstanding the presence of one or more additional components therebetween). For example, the sensor 30 may be engaged with the alert system 54 although other components are disposed therebetween.

While several embodiments have been described in detail, it will be apparent to those skilled in the art that the disclosed embodiments may be modified. Therefore, the foregoing description is to be considered exemplary rather than limiting.

What is claimed is:

1. A method for detecting at least partial medical device disengagement from a patient, the method comprising:
inserting the medical device in the patient so that a Hall effect sensor operatively connected to the medical device is operatively positioned at an initial location that is substantially proximate to one of a plurality of magnetically charged components that are removably attached to the patient, wherein each of the plurality of magnetically charged components is positioned a predetermined distance from an other of the plurality of magnetically charged components;
detecting, with the sensor, a magnetic field indicative of the one of the plurality of magnetically charged components;
monitoring, with the sensor, for a failure to detect the magnetic field indicative of the one of the plurality of magnetically charged components;
monitoring, with the sensor, for a subsequent detection of an other magnetic field indicative of an other of the plurality of magnetically charged components in response to the failure to detect the magnetic field indicative of the one of the plurality of the magnetically charged components; and
associating the subsequent detection with movement of the medical device substantially equal to the predetermined distance between the one of the plurality of magnetically charged components and the other of the plurality of the magnetically charged components.

2. The method as defined in claim 1, further comprising:
detecting a plurality of subsequent detections, each of the plurality of subsequent detections following a failure to detect a magnetic field of an other of the plurality of magnetically charged components; and
determining a distance of medical device disengagement based on the predetermined distance between the one of the plurality of magnetically charged components and the other of the plurality of magnetically charged components that corresponds with a final subsequent detection.

3. The method as defined in claim 1, further comprising:
transmitting an alert signal from the Hall effect sensor to an alert system when the movement of the medical device exceeds a predetermined threshold; and
emitting at least one of an audible alarm, a visual alarm, or a tactile alarm from the alert system in response to the alert signal.

4. The method as defined in claim 1 wherein the Hall effect sensor responds to a magnetic field having a predetermined minimum intensity.

5. The method as defined in claim 1, further comprising transmitting, from the Hall effect sensor to a processor, a signal indicative of the detected magnetic field, a signal indicative of the failure to detect the magnetic field, and a signal indicative of the subsequently detected magnetic field.

6. The method as defined in claim 1 wherein the medical device is a needle or a catheter.

7. The method as defined in claim 1 wherein prior to inserting the medical device, the method further comprises removably attaching each of the plurality of magnetically charged components to the patient via an adhesive bandage, an elastic band, a band configured with one or more of a hook, a snap, a button, a hook-and-loop fastener, or an adhesive, or combinations thereof.

8. A device configured to detect at least partial disengagement of a medical device from a patient, the device comprising:
a plurality of magnetically charged components configured to be operatively and removably attached to the patient, each of the plurality of the magnetically charged components positioned a predetermined distance from an other of the magnetically charged components;
a Hall effect sensor affixed to the medical device so that when the medical device is in an engagement position, the Hall effect sensor is substantially adjacent to one of the plurality of the magnetically charged components, wherein the sensor is configured to: i) detect a magnetic field of each of the magnetically charged components when the sensor is substantially proximate thereto, ii) recognize a failure to detect the magnetic field of each of the plurality of the magnetically charged components, and iii) transmit a signal representative of at least one of the detection or the failure to detect; and
a processor in operative communication with the sensor, the processor configured to monitor the signals, and to associate the detection of a magnetic field of an other of the plurality of magnetically charged components following the failure to detect a magnetic field of the one of the plurality of magnetically charged components with movement of the medical device substantially equal to a distance between the engagement position and a position of the other of the plurality of magnetically charged components.

9. The device as defined in claim 8 wherein the processor is configured to detect a plurality of subsequent detections, each of the plurality of the subsequent detections following a failure to detect a magnetic field of an other of the plurality of magnetically charged components, the processor further configured to determine a distance of medical device disengagement based on the predetermined distance between the one of the plurality of magnetically charged components and the other of the plurality of magnetically charged components that corresponds with a final subsequent detection.

10. The device as defined in claim 8, further comprising an alert system in operative communication with the Hall effect sensor, the alert system configured to receive an alert signal from the Hall effect sensor when the movement of the medical device exceeds a predetermined threshold.

11. The device as defined in claim 10 wherein the alert system is configured to emit at least one of an audible alarm, a visual alarm, or a tactile alarm in response to the alert signal.

12. The device as defined in claim 8 wherein the Hall effect sensor responds to a magnetic field having a predetermined minimum intensity.

13. The device as defined in claim 8 wherein the Hall effect sensor is configured to transmit to the processor a signal indicative of the detected magnetic field, a signal indicative of the failure to detect the magnetic field, and a signal indicative of the subsequently detected magnetic field.

14. The device as defined in claim 8 wherein the medical device is a needle or a catheter.

15. The device as defined in claim 8 wherein the plurality of the magnetically charged components is removably attachable via an adhesive bandage, an elastic band, a band configured with one or more of a hook, a snap, a button, a hook-and-loop fastener, or an adhesive, or combinations thereof.

16. A method for making a device configured to detect at least partial disengagement of a medical device from a patient, the method comprising:
operatively disposing a plurality of magnetically charged components on a removably attachable mechanism, each of the plurality of magnetically charged components positioned a predetermined distance from each of the other of the plurality of magnetically charged components;
affixing a Hall effect sensor to the medical device; and
positioning a medical device having the Hall effect sensor affixed thereto at an initial location so that the Hall effect sensor is substantially proximate to one of a plurality of magnetically charged components.

17. The method as defined in claim 16, further comprising operatively connecting the Hall effect sensor to a processor.

18. The method as defined in claim 16, further comprising operatively connecting the Hall effect sensor to an alert system.
